(19) United States
(12) Patent Application Publication
(10) Pub. No.: US 2014/0330078 A1
(43) Pub. Date: Nov. 6, 2014

(54) ENDOSCOPE AND IMAGE PROCESSING APPARATUS USING THE SAME

(71) Applicant: Samsung Electronics Co., Ltd., Suwon-si (KR)

(72) Inventors: Won Jun HWANG, Seoul (KR); Kyung Shik Roh, Seongnam-si (KR); Young Bo Shim, Seoul (KR); Suk June Yoon, Seoul (KR)

(73) Assignee: Samsung Electronics Co., Ltd., Suwon-si (KR)

(21) Appl. No.: 14/031,417

(22) Filed: Sep. 19, 2013

(30) Foreign Application Priority Data
May 3, 2013 (KR) 10-2013-0050186

(51) Int. Cl. A61B 1/00

(52) U.S. Cl. CPC A61B 1/00174; A61B 1/00193
USPC 600/111; 600/109

(57) ABSTRACT

An endoscope to acquire a 3D image and a wide view-angle image and an image processing apparatus using the endoscope includes a front image acquirer to acquire a front image and a lower image acquirer to acquire a lower image in a downward direction of the front image acquirer. The front image acquirer includes a first objective lens and a second objective lens arranged side by side in a horizontal direction. The lower image acquirer includes a third objective lens located below the first objective lens and inclined from the first objective lens and a fourth objective lens located below the second objective lens and inclined from the second objective lens.

### CROSS-REFERENCE TO RELATED APPLICATIONS

This application claims the priority benefit of Korean Patent Application No. 10-2013-0050186, filed on May 3, 2013 in the Korean Intellectual Property Office, the disclosure of which is incorporated herein by reference.

### BACKGROUND

1. Field

The following description relates to an endoscope that may acquire a 3-Dimensional (3D) image and a wide view-angle image, and an image processing apparatus using the endoscope.

2. Description of the Related Art

Minimally invasive surgery refers to surgical methods to minimize the size of an incision. While laparotomy uses relatively large surgical incisions through a part of a human body (e.g., the abdomen), in minimally invasive surgery, after forming at least one small port (incision or invasive hole) of 0.5 cm˜1.5 cm through the abdominal wall, an operator inserts a video camera and various surgical tools through the port, to perform surgery while viewing an image.

Compared to laparotomy, minimally invasive surgery has several advantages, such as low pain after surgery, early recovery, early restoration of ability to eat, short hospitalization, rapid return to daily life, and superior cosmetic effects due to a small incision. Accordingly, minimally invasive surgery has been used in gall resection, prostate cancer, and herniotomy operations, etc, and the use range thereof continues to expand.

Examples of surgical robots for use in minimally invasive surgery include a multi-port surgical robot and a single-port surgical robot. The multi-port surgical robot is configured to introduce a plurality of robotic surgical tools into the abdominal cavity of a patient through individual incisions. On the other hand, the single-port surgical robot is configured to introduce a plurality of robotic surgical tools into the abdominal cavity of a patient through a single incision.

In the case of surgery using the multi-port surgical robot or the single-port surgical robot, an endoscope is inserted into the abdominal cavity of the patient to capture an image of the interior of the abdominal cavity of the patient using the endoscope. The captured image is provided to an operator.

The multi-port surgical robot or the single-port surgical robot, adapted to capture an image of the interior of the abdominal cavity of the patient through the endoscope, may have difficulty in securing the operator's view when compared to laparotomy.

### SUMMARY

It is an aspect of the present disclosure to provide an endoscope that may acquire a 3D image and a wide view-angle image, and an image processing apparatus using the endoscope.

Additional aspects of the disclosure will be set forth in part in the description which follows and, in part, will be obvious from the description, or may be learned by practice of the invention.

In accordance with an aspect of the disclosure, an endoscope includes a front image acquirer including a first objective lens and a second objective lens arranged side by side in a horizontal direction, the front image acquirer serving to acquire a front image, and a lower image acquirer including a third objective lens located below the first objective lens and inclined from the first objective lens and a fourth objective lens located below the second objective lens and inclined from the second objective lens, the lower image acquirer serving to acquire a lower image in a downward direction of the front image acquirer.

In accordance with an aspect of the disclosure, an image processing apparatus includes an endoscope including a front image acquirer to acquire a front image and a lower image acquirer to acquire a lower image in a downward direction of the front image acquirer, wherein the front image acquirer includes a first objective lens and a second objective lens arranged side by side in a horizontal direction, and the lower image acquirer includes a third objective lens located below the first objective lens and inclined from the first objective lens and a fourth objective lens located below the second objective lens and inclined from the second objective lens, and an image processor to generate a result image based on a plurality of images acquired via the endoscope.

In accordance with an aspect of the disclosure, a method of generating a combination image with an endoscope may include acquiring a front image through a front objective lens provided in a plane orthogonal to a central axis of the endoscope, acquiring a lower image through a lower objective lens provided to form an angle with the front objective lens such that a viewpoint of the front image is skewed from a viewpoint of the lower image, and generating the combination image based on the front image and the lower image.

The angle may be variable depending on a rotation of the lower objective lens.

### BRIEF DESCRIPTION OF THE DRAWINGS

The patent or application file contains at least one drawing executed in color. Copies of this patent or patent application publication with color drawings will be provided by the Office upon request and payment of the necessary fee. These and/or other aspects of the disclosure will become apparent and more readily appreciated from the following description of the embodiments, taken in conjunction with the accompanying drawings of which:

FIG. 1 is a perspective view of an endoscope according to an embodiment;

FIGS. 2A to 2C are front views of the endoscope shown in FIG. 1, illustrating embodiments with regard to arrangement of at least one light source;

FIG. 3 is a side sectional view of the endoscope shown in FIG. 1, showing an embodiment with regard to an internal configuration of the endoscope;

FIG. 4 is a side sectional view of the endoscope shown in FIG. 1, showing an embodiment with regard to the internal configuration of the endoscope;

FIG. 5 is a view showing a control configuration of an image processing apparatus according to an embodiment;

FIG. 6 is a view showing the operation sequence of the image processing apparatus according to an embodiment;

FIG. 7 is a perspective view of an endoscope according to an embodiment;

FIG. 8 is a side sectional view of the endoscope shown in FIG. 7, showing a state before a lower image acquirer is inclined from a front image acquirer;

FIG. 9 is a side sectional view of the endoscope shown in FIG. 7, showing a state after the lower image acquirer is inclined from the front image acquirer;

FIG. 10 is a view showing a control configuration of an image processing apparatus according to an embodiment;

FIG. 11 is a view showing the operation sequence of the image processing apparatus according to an embodiment;

FIG. 12A is a view exemplifying a plurality of images acquired via the endoscope of the image processing apparatus, and FIG. 12B is a view showing an image processed by the image processing apparatus;

FIG. 13 is a perspective view of an endoscope according to an embodiment;

FIG. 14 is a side sectional view of the endoscope shown in FIG. 13, showing a state before a lower image acquirer and an upper image acquirer are inclined from a front image acquirer; and

FIG. 15 is a side sectional view of the endoscope shown in FIG. 13, showing a state after the lower image acquirer and the upper image acquirer are inclined from the front image acquirer.

### DETAILED DESCRIPTION

Advantages and features of the embodiments of the present disclosure and methods to achieve the advantages and features will become apparent with reference to the following detailed description and embodiments described below in detail in conjunction with the accompanying drawings. However, the embodiments of the present disclosure are not limited to the embodiments that will be described hereinafter, and may be realized in various ways. Rather, these embodiments are provided so that this disclosure will be thorough and complete, and will fully convey the scope to those skilled in the art, and should be defined by the scope of the claims.

Reference will now be made in detail to an endoscope and an image processing apparatus using the endoscope according to the embodiments of the present disclosure, examples of which are illustrated in the accompanying drawings, wherein like reference numerals refer to like elements throughout.

The endoscope of the disclosure includes a front image acquirer and a lower image acquirer. The front image acquirer serves to acquire an image in front of the endoscope. The front image acquirer is comprised of a first image acquirer and a second image acquirer. The lower image acquirer serves to acquire a lower image, i.e. an image in a downward direction from the front image acquirer. The lower image acquirer is comprised of a third image acquirer and a fourth image acquirer.

Each of the first to fourth image acquirers may include a lens and an image sensor. All of the first to fourth image acquirers may be provided in a cable of the endoscope, or some of the image acquirers may be provided in the cable. The configuration of a tip end of the endoscope may differ according to whether or not all of the first to fourth image acquirers are provided in the cable of the endoscope. Hereinafter, an embodiment in which all of the first to fourth image acquirers are provided in the cable of the endoscope will be described. In addition, an embodiment in which some of the first to fourth image acquirers are provided in the cable of the endoscope will be described.

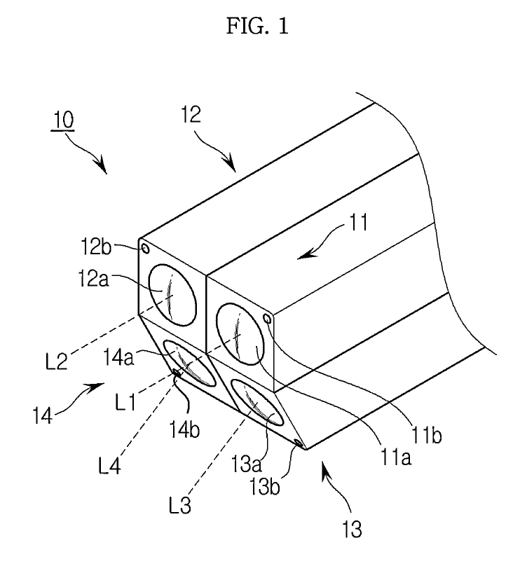

FIG. 1 is a perspective view of the endoscope 10 according to an embodiment.

Referring to FIG. 1, the endoscope 10 according to an embodiment includes all four image acquires 11, 12, 13, and 14 provided in a cable of the endoscope 10. Each of the image acquirers 11, 12, 13, or 14 may include an objective lens 11a, 12a, 13a, or 14a and an image sensor. In the following description, the four image acquirers 11, 12, 13, and 14 are respectively referred to as the first image acquirer 11, the second image acquirer 12, the third image acquirer 13, and the fourth image acquirer 14. In addition, components included in the respective image acquirers 11, 12, 13, and 14 are distinguished using terms ‘first’, ‘second’, ‘third’, and ‘fourth’.

A tip end of the endoscope 10 has a front face and a slope face. The slope face is tilted by a predetermined angle on the basis of the front face and is located below the front face.

A first objective lens 11a of the first image acquirer 11 and a second objective lens 12a of the second image acquirer 12 are horizontally arranged side by side at the front face. The first objective lens 11a serves to capture an image of a subject within a predetermined view angle (for example, 120 degrees) about an optical axis L1. Likewise, the second objective lens 12a serves to capture an image of the subject within a predetermined view angle about an optical axis L2.

A third objective lens 13a of the third image acquirer 13 and a fourth objective lens 14a of the fourth image acquirer 14 are horizontally arranged side by side at the slope face. The third objective lens 13a serves to capture an image of the subject within a predetermined view angle about an optical axis L3. Likewise, the fourth objective lens 14a serves to capture an image of the subject within a predetermined view angle about an optical axis L4. In an example, the view angles of the third objective lens 13a and the fourth objective lens 14a may be equal to those of the first objective lens 11a and the second objective lens 12a. In an example, the view angles of the third objective lens 13a and the fourth objective lens 14a may be greater than those of the first objective lens 11a and the second objective lens 12a.

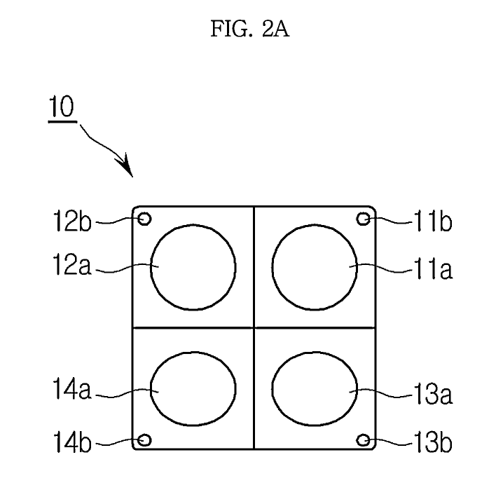

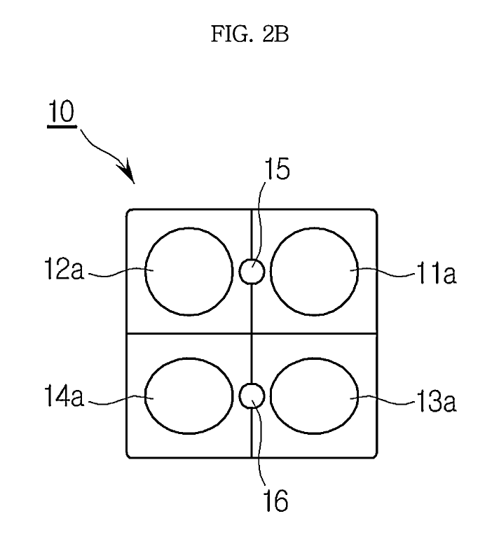

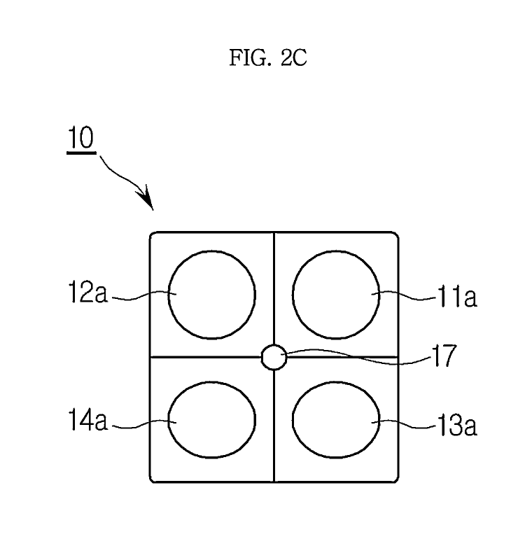

At least one light source 11b, 12b, 13b, or 14b is provided near the first to fourth objective lens 11a, 12a, 13a, and 14a. The at least one light source 11b, 12b, 13b, or 14b is forwardly oriented to emit light in the vicinity of the tip end of the endoscope 10. An example of the light source 11b, 12b, 13b, and 14b may include a Light Emitting Diode (LED). Various embodiments with regard to positioning of the at least one light source 11b, 12b, 13b, or 14b may be possible. A more detailed description thereof will follow with reference to FIGS. 2A to 2C.

FIG. 2A to 2C are front views of the endoscope 10 shown in FIG. 1, illustrating embodiments with regard to arrangement of at least one light source.

FIG. 2A shows a configuration in which the endoscope 10 includes a total of four light sources 11b, 12b, 13b, and 14b. In this case, the first to fourth light sources 11b, 12b, 13b, and 14b may be located respectively near the first to fourth objective lenses 11a, 12a, 13a, and 14a. For example, if the polygonal endoscope 10 has a square cross section, as exemplarily shown in FIG. 2A, the first to fourth light sources 11b, 12b, 13b and 14b may be provided at respective corners of the endoscope 10.

FIG. 2B shows a configuration in which the endoscope 10 includes a total of two light sources 15 and 16. In this case, the first light source 15 may be located between the first objective lens 11a and the second objective lens 12a. The second light source 16 may be located between the third objective lens 13a and the fourth objective lens 14a. However, positions of the first light source 15 and the second light source 16 are not limited to the above description. For example, the first light source 15 may be located in the front face of the endoscope 10 at a position above or below the position shown in FIG. 2B. Likewise, the second light source 16 may be located in the slope face of the endoscope 10 at a position above or below the position shown in FIG. 2B.

FIG. 2C shows a configuration in which the endoscope 10 includes a single light source 17. In this case, the light source 17 may be located at the center of the endoscope 10. In the case of providing the single light source 17, the brightness of the light source 17 may be controlled to be higher than that in the case of providing a plurality of light sources. In the following description, the case in which the endoscope 10 includes the four light sources 11b, 12b, 13b and 14b as exemplarily shown in FIG. 2A will be described by way of example.

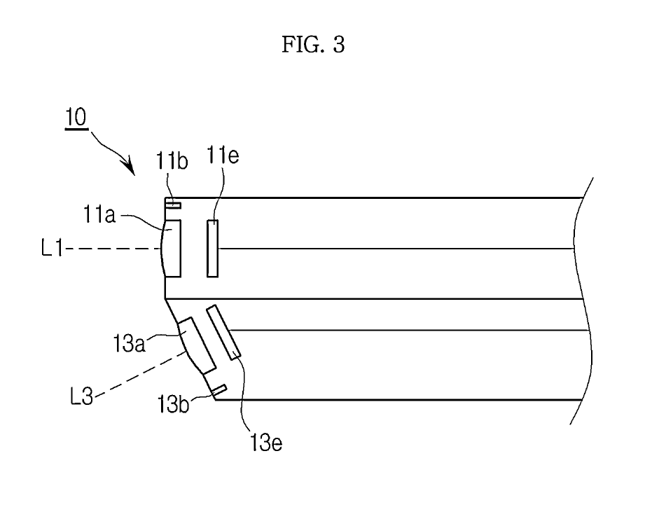

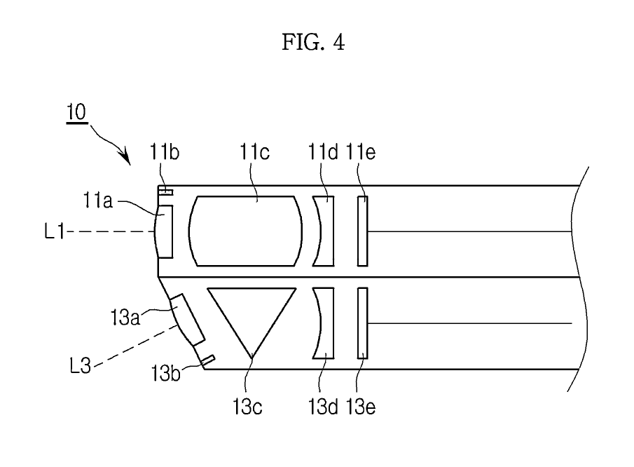

Next, an internal configuration of the endoscope 10 will be described with reference to FIGS. 3 and 4.

FIG. 3 is a side sectional view of the endoscope 10 shown in FIG. 1, showing an embodiment with regard to the internal configuration of the endoscope 10.

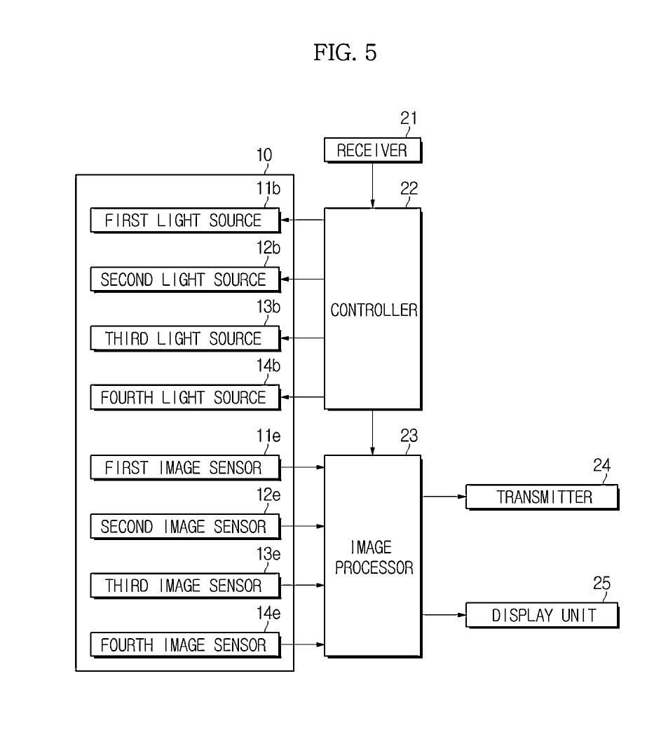

As exemplarily shown in FIG. 3, the first light source 11b is installed above the first objective lens 11a, and a first image sensor 11e is installed behind the first objective lens 11a. In this case, the first image sensor 11e is installed to face the first objective lens 11a. Although FIG. 3 shows only the internal configuration of the endoscope 10 behind the first objective lens 11a, the internal configuration of the endoscope 10 behind the second objective lens 12a has the same configuration as that behind the first objective lens 11a. That is, a second image sensor (see ‘12e’ of FIG. 5) is installed behind the second objective lens 12a to face the second objective lens 12a.

A third light source 13b is installed below the third objective lens 13a, and a third image sensor 13e is installed behind the third objective lens 13a. In this case, the third image sensor 13e is installed to face the third objective lens 13a. Although FIG. 3 shows only the internal configuration of the endoscope 10 behind the third objective lens 13a, the internal configuration of the endoscope 10 behind the fourth objective lens 14a has the same configuration as that behind the third objective lens 13a. That is, a fourth image sensor (see ‘14e’ of FIG. 5) is installed behind the fourth objective lens 14a to face the fourth objective lens 14a.

Meanwhile, examples of the image sensor may include a Charge Coupled Device (CCD) image sensor or a Complementary Metal Oxide Semiconductor (CMOS) image sensor.

The CCD image sensor may include an external lens, a micro lens, a color filter array, and a pixel array. If the CCD image sensor is placed in the endoscope 10, a timing generation IC, a timing regulation circuit, an Analog to Digital (A/D) converter, a CCD drive circuit, and the like may be additionally provided.

The CCD image sensor may include an external lens, a micro lens, a color filter array, a pixel array, an A/D converter to convert an analog signal read-out from the pixel array into a digital signal, and a digital signal processor to process the digital signal output from the A/D converter, all of which are provided on a single chip.

FIG. 4 is a side sectional view of the endoscope 10 shown in FIG. 1, showing another embodiment with regard to the internal configuration of the endoscope 10.

As exemplarily shown in FIG. 4, a group of first relay lenses 11c and 11d and the first image sensor 11e are arranged behind the first objective lens 11a. The first relay lens group consists of a plurality of lenses. FIG. 3 shows the case in which the first relay lens group includes a load lens 11c and a plane-concave lens 11d. The first relay lenses 11c and 11d assists light emitted from the first objective lens 11a in forming an image on the image sensor 11e. The image sensor 11e converts the formed image into electric signals.

Although FIG. 4 shows only the internal configuration of the endoscope 10 behind the first objective lens 11a, the internal configuration behind the second objective lens 12a is equal to the internal configuration behind the first objective lens 11a. That is, a group of second relay lenses (not shown) and the second image sensor (see ‘12e’ of FIG. 5) are arranged behind the second objective lens 12a.

A prism 13c, a third relay lens 13d, and the third image sensor 13e are arranged behind the third objective lens 13a. The prism 13c refracts light emitted from the third objective lens 13a. Refraction of light emitted from the third objective lens 13a serves to change the path of light toward the third image sensor 13e that is not oriented to face the third objective lens 13. The light refracted by the prism 13c is introduced into the relay lens 13d. The relay lens 13d assists light refracted by the prism 13c in forming an image on the third image sensor 13e. The third image sensor 13e converts the formed image into electric signals.

Although FIG. 4 shows only the internal configuration of the endoscope 10 behind the third objective lens 13a, the internal configuration behind the fourth objective lens 14a is equal to the internal configuration behind the third objective lens 13a.

As such, the outer appearance and the inner configuration of the endoscope 10 according to an embodiment will be described with reference to FIGS. 1 to 4. Although FIGS. 1 and 2C show the endoscope 10 as having a square cross section, this is exaggerated for explanation, and the cross section of the endoscope 10 may have another shape, such as a circular shape, for example.

FIGS. 3 and 4 show the case in which the image sensors 11e, 12e, 13e, and 14e are arranged to correspond to the respective objective lenses 11a, 12a, 13a, and 14a. However, a smaller number of the image sensors may be provided. In an example, a single image sensor (not shown) may be arranged in regions corresponding to the first to fourth objective lenses 11a, 12a, 13a, and 14a.

Next, the image processing apparatus to process an image acquired by the endoscope 10 will be described.

FIG. 5 is a view showing a control configuration of the image processing apparatus according to an embodiment.

As exemplarily shown in FIG. 5, the image processing apparatus may include the endoscope 10, a receiver 21, a controller 22, an image processor 23, a transmitter 24, and a display unit 25.

The endoscope 10 may include the first to fourth light sources 11b, 12b, 13b, and 14b, and the first to fourth image sensors 11e, 12e, 13e, and 14e as described above with reference to FIGS. 1 to 4.

The receiver 21 receives a control instruction. The control instruction may be transmitted from an external device (e.g., a master console of a surgical robot), or may be input by an operator via an input unit (not shown) provided in the image processing apparatus. Examples of the control instruction may include an instruction to control brightness of each light source 11b, 12b, 13b, or 14b and an instruction to activate the image processor 23.

The controller 22 controls brightness of each light source 11b, 12b, 13b, or 14b and activates the image processor 23 in response to a control instruction received via the receiver 21.

The image processor 23 generates an output image based on images acquired via the first to fourth image sensors 11e, 12e, 13e, and 14e. Examples of the output images may include a wide view-angle image and a 3D image of regions in front of and below the endoscope 10.

The image processor 23 matches the images acquired via the first to fourth image sensors 11e, 12e, 13e, and 14e to generate a wide view-angle image. More specifically, the image processor 23 extracts at least one feature from each of the images acquired via the first to fourth image sensors 11e, 12e, 13e, and 14e. An example of a feature extraction algorithm may include Scale Invariant Feature Transform (SIFT). The SIFT is an algorithm for extraction of features that are invariant translation, rotation, and rescaling of an image. The SIFT is known technology and a detailed description thereof will be omitted. If at least one feature is extracted from each image, the image processor 23 matches the images based on the extracted at least one feature. As a result, a wide view-angle image in a range of 180 degrees or more is generated.

The image processor 23 may generate a 3D image based on the images acquired via the first to fourth image sensors 11e, 12e, 13e, and 14e. The 3D image may be generated based on a left-eye image and a right-eye image.

In this case, the left-eye image and the right-eye image may be generated by the following method. The image processor 23 generates a left-eye image based on the image acquired by the first image sensor 11e and the image acquired by the third image sensor 13e. In addition, the image processor 23 generates a right-eye image based on the image acquired by the second image sensor 12e and the image acquired by the fourth image sensor 14e. Through generation of the 3D image based on the left-eye image and the right-eye image using the above-described method, a 3D image viewed at an angle including regions in front of and below the endoscope 10 may be acquired.

The transmitter 24 may transmit at least one of the 3D image and the wide view-angle image generated by the image processor 23 to an external device (for example, the master console of the surgical robot).

The display unit 25 may display at least one of the 3D image and the wide view-angle image that are generated by the image processor 23. A plurality of display units 25 may be provided. In this case, display regions of the respective display units 25 may display different images. Alternatively, a single image may be displayed on the entire display region of the plurality of display units 25. The display units 25, for example, may be a Cathode Ray Tube (CRT), Liquid Crystal Display (LCD), Light Emitting Diode (LED), Organic Light Emitting Diode (OLED), or Plasma Display Panel (PDP).

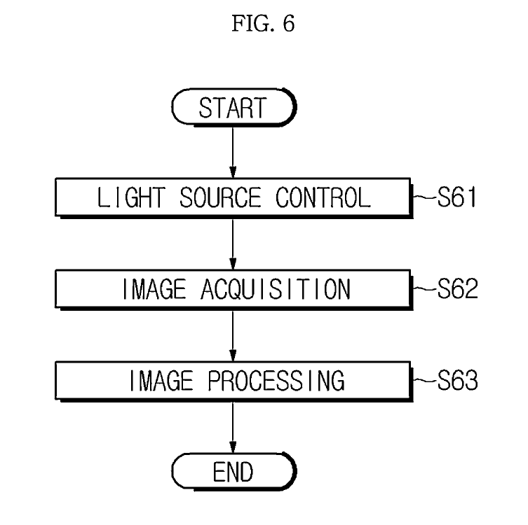

FIG. 6 is a view showing the operation sequence of the image processing apparatus according to an embodiment.

If the image processing apparatus receives a control instruction, brightness of a plurality of light sources is controlled according to the received control instruction (operation S61).

Under control of the brightness of the plurality of light sources, light reflected from a subject is introduced into the first to fourth objective lenses 11a, 12a, 13a, and 14a, and in turn the light emitted from the first to fourth objective lenses 11a, 12a, 13a, and 14a forms images on the first to fourth image sensors 11e, 12e, 13e, and 14e. Then, the first to fourth image sensors 11e, 12e, 13e, and 14e convert the formed images into electric signals. As a result, a plurality of images is acquired (operation S62).

Once the plurality of images has been acquired, processing of the plurality of acquired images is performed (operation S63). The image processing operation (operation S63) may include generating a wide view-angle image and generating a 3D image.

Generation of the wide view-angle image includes extracting at least one feature from each of the images acquired via the first to fourth image sensors 11e, 12e, 13e, and 14e, and matching the images based on the at least one extracted feature to generate a wide view-angle image.

Generation of the 3D image includes generating a left-eye image based on the image acquired via the first image sensor 11e and the image acquired via the third image sensor 13e, and generating a right-eye image based on the image acquired via the second image sensor 12e and the image acquired via the fourth image sensor 14e.

At least one of the wide view-angle image and the 3D image generated in the image processing operation S63 may be displayed via the display unit 25 of the image processing apparatus, or may be transmitted to the external device.

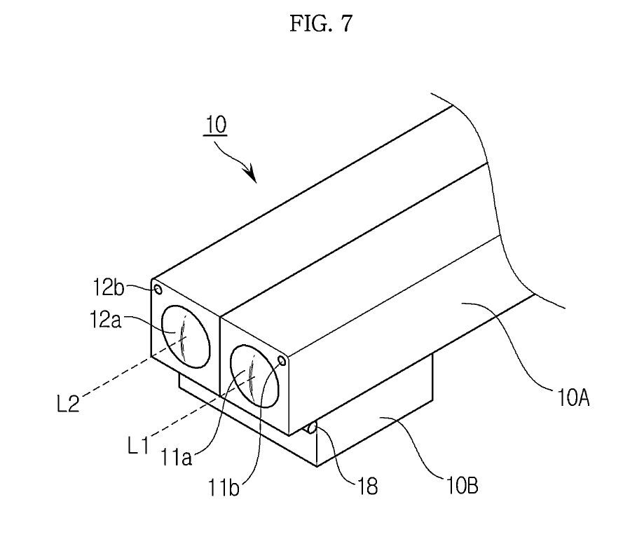

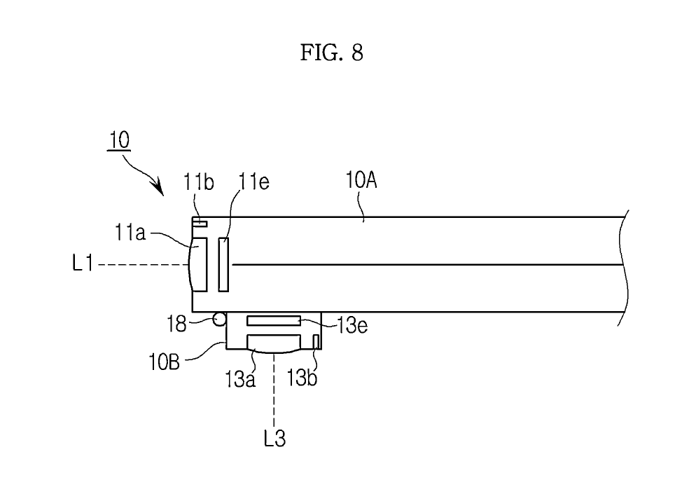

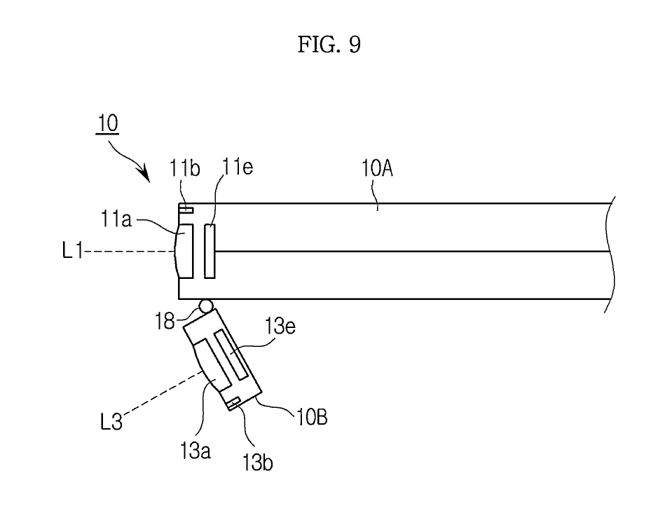

FIG. 7 is a perspective view of an endoscope according to an embodiment, and FIGS. 8 and 9 are side sectional views of the endoscope shown in FIG. 7.

Referring to FIGS. 7 to 9, the endoscope 10 includes a front image acquirer 10A and a lower image acquirer 10B.

The front image acquirer 10A serves to acquire an image in front of the endoscope 10 and is provided in the cable of the endoscope 10. The front image acquirer 10A includes the first image acquirer and the second image acquirer.

The lower image acquirer 10B serves to acquire an image below the front image acquirer 10A and is provided outside the cable. The lower image acquirer 10B includes the third image acquirer and the fourth image acquirer.

More specifically, the first objective lens 11a of the first image acquirer and the second objective lens 12a of the second image acquirer are horizontally arranged side by side at the front face of the endoscope 10. The first image sensor 11e is arranged behind the first objective lens 11a to face the first objective lens 11a. Although not shown in the drawing, the second image sensor is arranged behind the second objective lens 12a to face the second objective lens 12a. In addition, as exemplarily shown in FIG. 8, the third image sensor 13e is arranged behind the third objective lens 13a to face the third objective lens 13a. The fourth image sensor is arranged behind the fourth objective lens 14a.

The first to fourth light sources 11b, 12b, 13b, and 14b are installed respectively near the first to fourth image acquirers. The respective light sources 11b, 12b, 13b, and 14b emit light in the vicinity of the endoscope 10.

A joint 18 is provided between the front image acquirer 10A and the lower image acquirer 10B. A drive unit 19, such as a motor, is provided at the joint 18. The drive unit 19 is operated in response to a control signal to pivotally rotate the joint 18 upward or downward. As the joint 18 is pivotally rotated upward or downward, the lower image acquirer 10B is also pivotally rotated about a coupling shaft.

The lower image acquirer 10B normally remains completely folded to come into contact with the cable of the endoscope 10 as exemplarily shown in FIG. 8. That is, the optical axis L1 of the first objective lens 11a and the optical axis L3 of the third objective lens 13a maintain an angle of 90 degrees. The endoscope 10 is inserted into an incision of the patient in such a state, or is moved along a guide tube (not shown) previously inserted into the incision. This may reduce a cross sectional area of the tip end of the endoscope 10, which may reduce damage to the incision by the endoscope 10 when the endoscope 10 is inserted into the incision. In addition, maneuverability of the endoscope 10 may be ensured when the endoscope 10 is moved along the guide tube.

Once the endoscope 10 has been inserted into the abdominal cavity of the patient, drive power is applied to the drive unit 19 provided at the joint 18 to operate the drive unit 19. As a result, as exemplarily shown in FIG. 9, the lower image acquirer 10B is moved and inclined from the front image acquirer 10B under control. If the inclination of the lower image acquirer 10B is controlled such that an angle between the optical axis L1 of the first objective lens 11a and the optical axis L3 of the third objective lens 13a is less than 90 degrees, the image capture range of the first objective lens 11a and the image capture range of the third objective lens 13a overlap each other. Although not shown in the drawings, the image capture range of the second objective lens 12a overlaps with the image capture range of the fourth objective lens 14a. As a result, a wide view-angle image with regard to regions in front of and below the endoscope 10 may be acquired.

Next, the image processing apparatus to process the image acquired by the endoscope 10 as described above will be described.

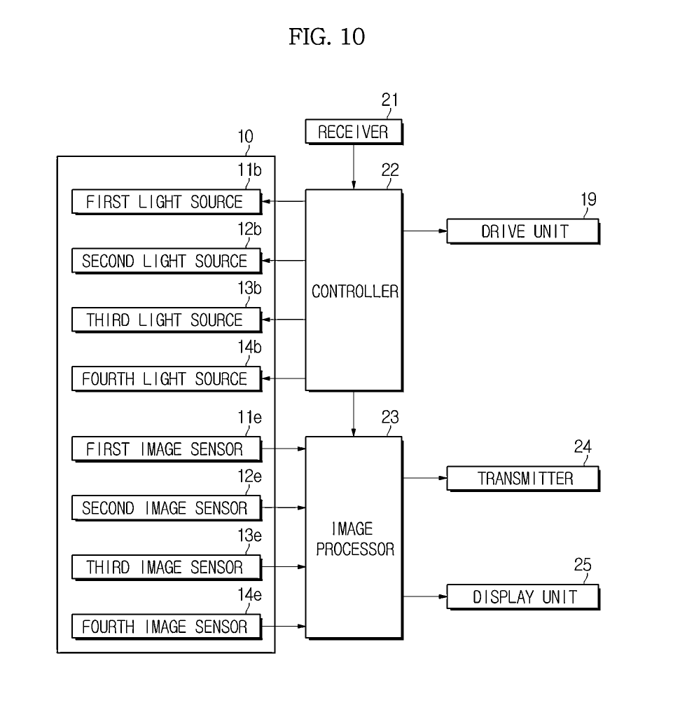

FIG. 10 is a view showing a control configuration of the image processing apparatus according to an embodiment.

As exemplarily shown in FIG. 10, the image processing apparatus may include the endoscope 10, the receiver 21, the controller 22, the drive unit 19, the image processor 23, the transmitter 24, and the display unit 25.

The endoscope 10 may include the first to fourth light sources 11b, 12b, 13b, and 14b, and the first to fourth image sensors 11e, 12e, 13e, and 14e as described above with reference to FIGS. 7 to 9.

The receiver 21 receives a control instruction. The control instruction may be transmitted from the external device, or may be input by the operator via the input unit (not shown) provided in the image processing apparatus. Examples of the control instruction may include an instruction to control the inclination of the lower image acquirer 10B with respect to the front image acquirer 10A, an instruction to control brightness of each light source, and an instruction to activate the image processor 23.

The controller 22 applies drive power to the drive unit 19 in response to the control instruction received via the receiver 21 to enable control of the inclination of the lower image acquirer 10B with respect to the front image acquirer 10A. In addition, the controller 22 controls brightness of each light source 11b, 12b, 13b, or 14b and activates the image processor 23 in response to the received control instruction.

The drive unit 19 is operated in response to the control signal of the controller 22 to rotate the joint 18 provided between the front image acquirer 10A and the lower image acquirer 10B. As a result, an angle between the front image acquirer 10A and the lower image acquirer 10B is controlled.

The image processor 23 generates an output image based on the images acquired via the first to fourth image sensors 11e, 12e, 13e, and 14e. Examples of the output image may include a wide view-angle image, and 3D images in front of and below the endoscope 10.

The wide view-angle image may be generated by extracting at least one feature of each of the images acquired via the first to fourth image sensors 11e, 12e, 13e, and 14e and matching the images based on the at least one extracted feature.

The 3D image of regions in front of and below the endoscope 10 may be generated based on a left-eye image and a right-eye image. In this case, the left-eye image may be generated by matching the image acquired by the first image sensor 11e with the image acquired by the third image sensor 13e. The right-eye image may be generated by matching the image acquired by the second image sensor 12e with the image acquired by the fourth image sensor 14e.

The transmitter 24 may transmit at least one of the 3D image and the wide view-angle image generated by the image processor 23 to the external device.

The display unit 25 may display at least one of the 3D image and the wide view-angle image that are generated by the image processor 23. A plurality of display units 25 may be provided. In this case, display regions of the respective display units 25 may display different images. Alternatively, a single image may be displayed on the entire display region of the plurality of display units 25. The display method is determined according to the user selection.

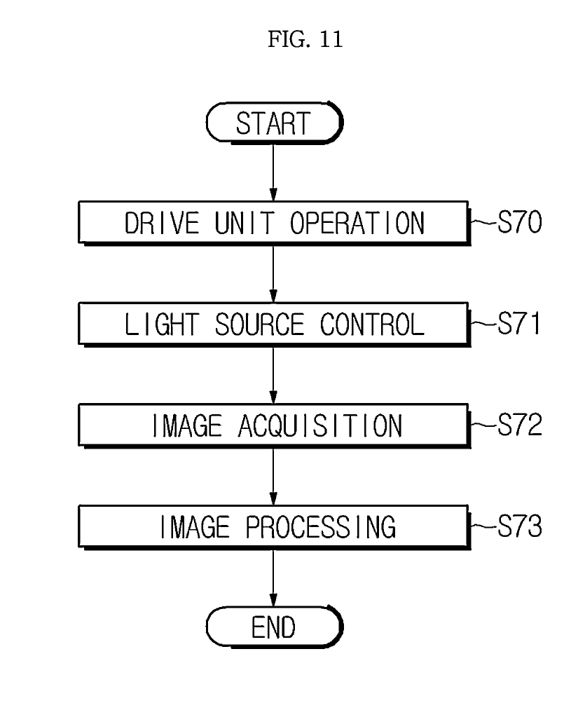

FIG. 11 is a view showing the operation sequence of the image processing apparatus according to an embodiment.

For the description below, it is assumed that the endoscope has been inserted into the abdominal cavity of the patient.

If the image processing apparatus receives a control instruction, the drive unit 19 is operated in response to the received control instruction (operation S70). As a result, as the joint 18 is rotated about a coupling shaft, the inclination of the lower image acquirer 10B with respect to the front image acquirer 10A is controlled. That is, the angle between the front image acquirer 10A and the lower image acquirer 10B is controlled.

Thereafter, brightness of the plurality of light sources 11b, 12b, 13b, and 14b is controlled in response to the received instruction (operation S71).

If brightness of the plurality of light sources 11b, 12b, 13b, and 14b is controlled, light reflected from tissue inside the abdominal cavity is introduced into the first to fourth objective lenses 11a, 12a, 13a, and 14a, and the light emitted from the first to fourth objective lenses 11a, 12a, 13a, and 14a forms images on the first to fourth image sensors 11e, 12e, 13e, and 14e. Then, the first to fourth image sensors 11e, 12e, 13e, and 14e convert the formed images into electric signals. As a result, a plurality of images is acquired (operation S72).

Once the plurality of images has been acquired, processing of the plurality of images is performed (operation S73). The image processing operation S73 may include at least one of generating a wide view-angle image and generating a 3D image.

Generation of the wide view-angle image includes extracting features from each of the images acquired via the first to fourth image sensors 11e, 12e, 13e, and 14e, and matching the images based on the extracted features to generate a wide view-angle image.

Generation of the 3D image includes generating a left-eye image based on the image acquired via the first image sensor 11e and the image acquired via the third image sensor 13e, and generating a right-eye image based on the image acquired via the second image sensor 12e and the image acquired via the fourth image sensor 14e.

At least one of the wide view-angle image and the 3D image generated in the image processing operation S73 may be displayed via the display unit 25 of the image processing apparatus, or may be transmitted to the external device.

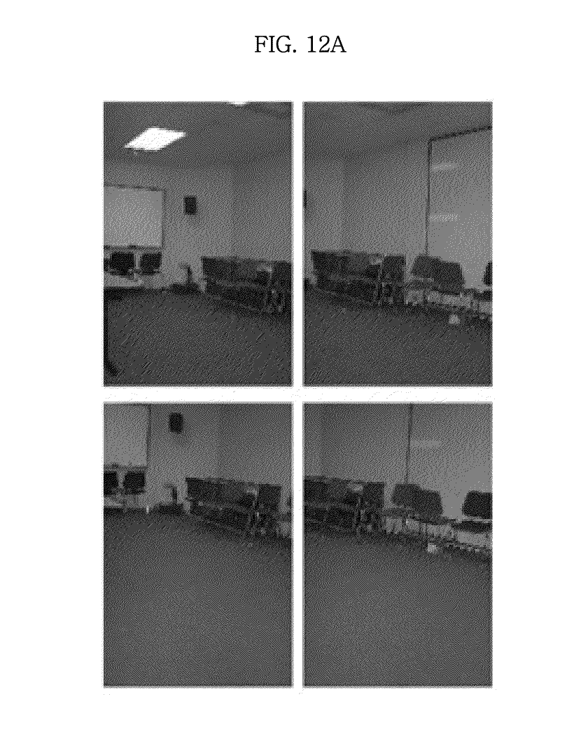

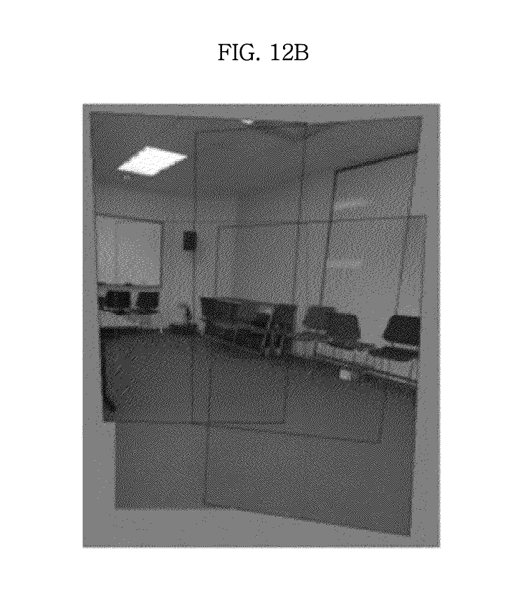

FIG. 12A is a view showing a plurality of images acquired via the endoscope 10 of the image processing apparatus, and FIG. 12B is a view showing an image processed by the image processing apparatus, i.e. an image acquired by matching the images shown in FIG. 12A.

As described above, the image processing apparatus extracts at least one feature from each of the plurality of images acquired via the endoscope 10 as exemplarily shown in FIG. 12A and matches the plurality of images based on the at least one extracted feature. As a result, a wide view-angle image as exemplarily shown in FIG. 12B is generated.

In addition to matching the images based on the at least one feature extracted from each of the plurality of images, the plurality of images may be matched based on mechanical properties of each image acquirer. For example, the plurality of images may be matched based on at least one parameter associated with the objective lens of each image acquirer, thereby generating a wide view-angle image.

Thereafter, the image processing apparatus may perform post-processing on the generated wide view-angle image. For example, in the wide view-angle image as exemplarily shown in FIG. 12B, a rim region, i.e. a portion where no image information is present is blacked out or deleted. As occasion demands, a process of enlarging the wide view-angle image by the deleted region or of moving the wide view-angle image may be performed.

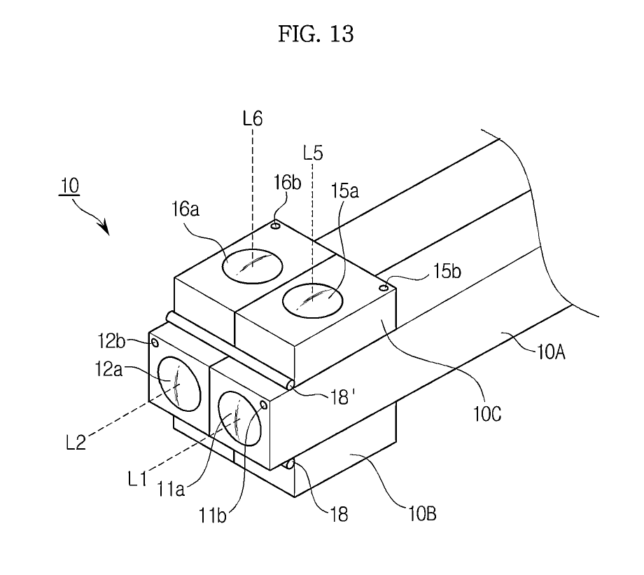

The endoscope to acquire front and lower images and the image processing apparatus including the endoscope have been described above. In an embodiment, the endoscope to acquire an upper image as well as front and lower images will be described with reference to FIGS. 13 to 15.

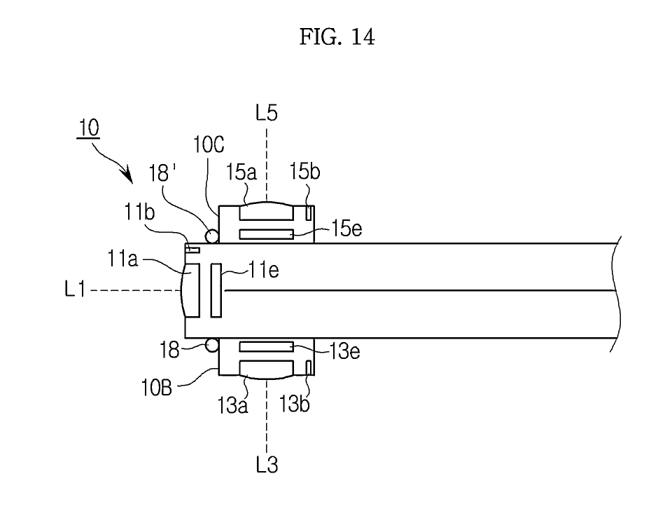

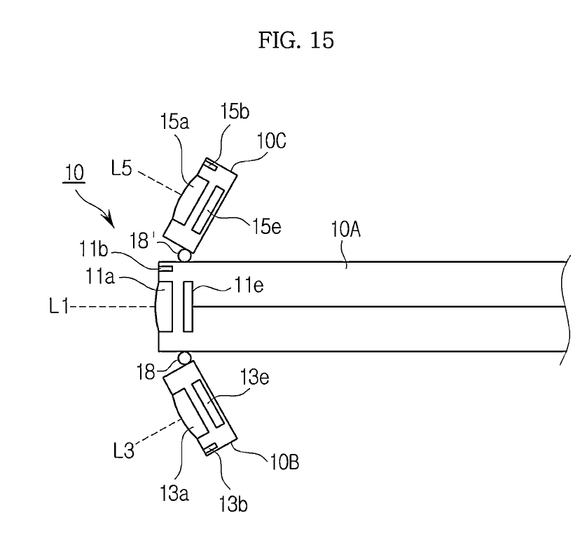

FIG. 13 is a perspective view of the endoscope according to an embodiment, and FIGS. 14 and 15 are side sectional views of the endoscope shown in FIG. 13.

Compared to the endoscope 10 as exemplarily shown in FIG. 7, the endoscope 10 as exemplarily shown in FIG. 13 further includes an upper image acquirer 10C provided outside the cable. The upper image acquirer 10C serves to acquire an upper image, i.e. an image in an upward direction from the front image acquirer 10A. The upper image acquirer 10C includes a fifth image acquirer and a sixth image acquirer.

More specifically, the first objective lens 11a of the first image acquirer and the second objective lens 12a of the second image acquirer are horizontally arranged side by side in front of the endoscope 10.

As exemplarily shown in FIG. 13, the first image sensor 11e is arranged behind the first objective lens 11a to face the first objective lens 11a. The third image sensor 13e is arranged behind the third objective lens 13a to face the third objective lens 13a. A fifth image sensor 15e is arranged behind a fifth objective lens 15a to face the fifth objective lens 15a.

Although not shown in FIG. 14, the second image sensor is arranged below the second objective lens 12a to face the second objective lens 12a. The fourth image sensor 14e is arranged behind the fourth objective lens 14a to face the fourth objective lens 14a. Likewise, a sixth image sensor is arranged behind a sixth objective lens 16a to face the sixth objective lens 16a.

The first to sixth light sources 11b, 12b, 13b, 14b, 15b, and 16b are respectively installed near the first to sixth image acquirers. The respective light sources 11b, 12b, 13b, 14b, 15b, and 16b emit light in the vicinity of the endoscope 10.

The joint 18 is provided between the front image acquirer 10A and the lower image acquirer 10B. In addition, a joint 18′ is provided between the front image acquirer 10A and the upper image acquirer 10C. Drive units (not shown), such as motors, are provided respectively at the joints 18 and 18′. The drive units are operated in response to a control signal to pivotally rotate the joints 18 and 18′ respectively. As the joints 18 and 18′ are pivotally rotated upward or downward, the lower image acquirer 10B and the upper image acquirer 10C are also pivotally rotated about respective coupling shafts.

The lower image acquirer 10B and the upper image acquirer 10C normally remains completely folded to come into contact with the cable of the endoscope 10 as exemplarily shown in FIG. 14. That is, the optical axis L1 of the first objective lens 11a and the optical axis L3 of the third objective lens 13a maintain an angle of 90 degrees, and the optical axis L1 of the first objective lens 11a and an optical axis L5 of the fifth objective lens 15a maintain an angle of 90 degrees. The endoscope 10 is inserted into an incision of the patient in such a state, or is moved along a guide tube (not shown) previously inserted into the incision. This may reduce a cross sectional area of the tip end of the endoscope 10, which may reduce damage to the incision by the endoscope 10 when the endoscope 10 is inserted into the incision. In addition, maneuverability of the endoscope 10 may be ensured when the endoscope 10 is moved along the guide tube.

Once the endoscope 10 has been inserted into the abdominal cavity of the patient, drive power is applied to the drive units provided respectively at the joints 18 and 18′ to operate the drive units. As a result, as exemplarily shown in FIG. 15, the lower image acquirer 10B is moved and inclined from the front image acquirer 10B under control and the upper image acquirer 10C is moved and inclined from the front image acquirer 10B under control.

If the inclination of the lower image acquirer 10B is controlled such that an angle between the optical axis L1 of the first objective lens 11a and the optical axis L3 of the third objective lens 13a is less than 90 degrees, the image capture range of the first objective lens 11a and the image capture range of the third objective lens 13a overlap each other. Although not shown in the drawings, the image capture range of the second objective lens 12a overlaps with the image capture range of the fourth objective lens 14a. As a result, a wide view-angle image with regard to regions in front of and below the endoscope 10 may be acquired.

If the inclination of the upper image acquirer 10C is controlled such that an angle between the optical axis L1 of the first objective lens 11a and the optical axis L5 of the fifth objective lens 15a is less than 90 degrees, the image capture range of the first objective lens 11a and the image capture range of the fifth objective lens 15a overlap each other. Although not shown in the drawings, the image capture range of the second objective lens 12a overlaps with the image capture range of the sixth objective lens 16a, which serves to capture an image of the subject within a predetermined view angle about an optical axis L6. As a result, a wide view-angle image with regard to regions in front of and below the endoscope 10 may be acquired.

As is apparent from the above description, it may be possible to acquire an image below an endoscope as well as an image in front of the endoscope.

Acquisition of a wide view-angle image including the image below the endoscope as well as the image in front of the endoscope may be accomplished, which may prevent a robotic surgical tool located below the endoscope from deviating from the operator's view and damaging organs or blood vessels.

The above-described embodiments may be recorded in computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter. The above-described devices may be configured to act as one or more software modules in order to perform the operations of the above-described embodiments, or vice versa.

Although the embodiments of the present disclosure have been shown and described, it would be appreciated by those skilled in the art that changes may be made in these embodiments without departing from the principles and spirit of the invention, the scope of which is defined in the claims and their equivalents.

What is claimed is:

1. An endoscope comprising:
a front image acquirer comprising a first objective lens and a second objective lens arranged side by side in a horizontal direction, the front image acquirer serving to acquire a front image; and
a lower image acquirer comprising a third objective lens located below the first objective lens and inclined from the first objective lens and a fourth objective lens located below the second objective lens and inclined from the second objective lens, the lower image acquirer serving to acquire a lower image in a downward direction of the front image acquirer.

2. The endoscope according to claim 1, wherein a tip end of the endoscope comprises a front face and a slope face tilted by a predetermined angle on the basis of the front face.

3. The endoscope according to claim 2,
wherein the first objective lens and the second objective lens are horizontally arranged at the front face of the tip end, and
wherein the third objective lens and the fourth objective lens are horizontally arranged at the slope face of the tip end.

4. The endoscope according to claim 3, wherein a first image sensor, a second image sensor, a third image sensor, and a fourth image sensor are respectively provided behind the first objective lens, the second objective lens, the third objective lens, and the fourth objective lens such that light emitted from the first objective lens, the second objective lens, the third objective lens, and the fourth objective lens forms images on the first image sensor, the second image sensor, the third image sensor, and the fourth image sensor, respectively.

5. The endoscope according to claim 4,
wherein a first relay lens is disposed between the first objective lens and the first image sensor such that light emitted from the first objective lens forms an image on the first image sensor, and
wherein a second relay lens is disposed between the second objective lens and the second image sensor such that light emitted from the second objective lens forms an image on the second image sensor.

6. The endoscope according to claim 4, wherein a prism to refract light emitted from the third objective lens and a relay lens to assist the light refracted by the prism in forming an image on the third image sensor are arranged in sequence between the third objective lens and the third image sensor.

7. The endoscope according to claim 1,
wherein the front image acquirer is provided inside a cable of the endoscope, and
wherein the lower image acquirer is provided outside the cable of the endoscope.

8. The endoscope according to claim 7, further comprising:
a joint provided between the front image acquirer and the lower image acquirer; and
a drive unit provided at the joint to rotate the joint.

9. The endoscope according to claim 1, further comprising at least one light source installed near at least one of the first objective lens, the second objective lens, the third objective lens, and the fourth objective lens.

10. An image processing apparatus comprising:
an endoscope comprising a front image acquirer to acquire a front image and a lower image acquirer to acquire a lower image in a downward direction of the front image acquirer, wherein the front image acquirer comprises a first objective lens and a second objective lens arranged side by side in a horizontal direction, and the lower image acquirer comprises a third objective lens located below the first objective lens and inclined from the first objective lens and a fourth objective lens located below the second objective lens and inclined from the second objective lens; and
an image processor to generate a result image based on a plurality of images acquired via the endoscope.

11. The apparatus according to claim 10,
wherein a tip end of the endoscope comprises a front face and a slope face tilted by a predetermined angle on the basis of the front face,
wherein the first objective lens and the second objective lens are horizontally arranged at the front face of the tip end, and
wherein the third objective lens and the fourth objective lens are horizontally arranged at the slope face of the tip end.

12. The apparatus according to claim 11, wherein a first image sensor, a second image sensor, a third image sensor, and a fourth image sensor are respectively provided behind the first objective lens, the second objective lens, the third objective lens, and the fourth objective lens such that light emitted from the first objective lens, the second objective lens, the third objective lens, and the fourth objective lens forms images on the first image sensor, the second image sensor, the third image sensor, and the fourth image sensor, respectively.

13. The apparatus according to claim 12,
wherein a first relay lens is disposed between the first objective lens and the first image sensor such that light emitted from the first objective lens forms an image on the first image sensor, and
wherein a second relay lens is disposed between the second objective lens and the second image sensor such that light emitted from the second objective lens forms an image on the second image sensor.

14. The apparatus according to claim 12, wherein a prism to refract light emitted from the third objective lens and a relay lens to assist the light refracted by the prism in forming an image on the third image sensor are arranged in sequence between the third objective lens and the third image sensor.

15. The apparatus according to claim 12, wherein the result image comprises at least one of a wide view-angle image and a 3-Dimensional (3D) image.

16. The apparatus according to claim 15, wherein the image processor extracts at least one feature from each of images acquired by the first image sensor, the second image sensor, the third image sensor, and the fourth image sensor, and matches the acquired images based on the at least one extracted feature, to form the wide view-angle image.

17. The apparatus according to claim 15, wherein the image processor generates a left-eye image based on the image acquired by the first image sensor and the image acquired by the third image sensor, generates a right-eye image based on the image acquired by the second image sensor and the image acquired by the fourth image sensor, and generates the 3D image based on the left-eye image and the right-eye image.

18. The apparatus according to claim 10,
wherein the front image acquirer is provided inside a cable of the endoscope, and the lower image acquirer is provided outside the cable of the endoscope, and
wherein a joint is provided between the front image acquirer and the lower image acquirer and is rotated by a drive unit.

19. A method of generating a combination image with an endoscope, the method comprising:
acquiring a front image through a front objective lens provided in a plane orthogonal to a central axis of the endoscope;
acquiring a lower image through a lower objective lens provided to form an angle with the front objective lens such that a viewpoint of the front image is skewed from a viewpoint of the lower image; and
generating the combination image based on the front image and the lower image.

20. The method of claim 19, wherein the angle is variable depending on a rotation of the lower objective lens.
